(19) United States
(12) Patent Application Publication
(10) Pub. No.: US 2025/0361539 A1
(43) Pub. Date: Nov. 27, 2025

(54) XANTHOMONAS CAMPESTRIS FOR FERMENTATIVE PRODUCTION OF TRANSPARENT XANTHAN GUM AND USE THEREOF

(71) Applicant: INNER MONGOLIA UNIVERSITY OF TECHNOLOGY, HUHHOT (CN)

(72) Inventors: ZHANYING LIU, HUHHOT (CN); HAN SUN, HUHHOT (CN)

(21) Appl. No.: 19/296,224

(22) Filed: Aug. 11, 2025

Related U.S. Application Data
(63) Continuation of application No. PCT/CN2024/136285, filed on Dec. 3, 2024.

(30) Foreign Application Priority Data
Dec. 21, 2023 (CN) 202311763510.X

(51) Int. Cl. C12P 19/06; C12N 1/20; C12R 1/64

(52) U.S. Cl. CPC C12P 19/06; C12N 1/205; C12R 2001/64

(57) ABSTRACT

Xanthomonas campestris for fermentative production of transparent xanthan gum and a method for using Xanthomonas campestris are provided. The Xanthomonas campestris is Xanthomonas campestris F417-6, deposited in Guangdong Province Microbiological Culture Collection Center on May 12, 2023, with a deposit number of GDMCC NO: 63459. The method includes preparing xanthan gum by performing fermentation using the Xanthomonas campestris as a fermentation strain.

### CROSS REFERENCE TO RELATED APPLICATIONS

The present disclosure is a continuation application of international application PCT/CN2024/136285 filed on Dec. 3, 2024, which international application claims the priority to Chinese patent application CN202311763510X, entitled “XANTHOMONAS CAMPESTRIS FOR FERMENTATIVE PRODUCTION OF TRANSPARENT XANTHAN GUM AND USE THEREOF” and filed on Dec. 21, 2023 with the Chinese Patent Office, the contents of which are incorporated herein by reference in their entirety.

### TECHNICAL FIELD

The present disclosure belongs to the technical field of microbiology, and particularly relates to Xanthomonas campestris for fermentative production of transparent xanthan gum and use thereof.

### BACKGROUND ART

Xanthan gum, also called yellow gum and xanthic gum, is a water-soluble microbial extracellular polysaccharide produced by aerobic fermentation of Xanthomonas campestris with carbohydrates as the main raw materials, which is currently the microbial polysaccharide with the largest production scale in the world. Due to its special macromolecular structure and colloidal properties, xanthan gum has excellent physical and chemical properties, such as suspension property, emulsification property, thickening property, pseudoplasticity, thermal stability, etc., may be widely used in various fields as a thickener, emulsifier, stabilizer, gelling agent, impregnation agent, film forming agent, etc., and is one of the most superior biological gums. Xanthan gum has the general properties of long-chain polymers, but contains more functional groups than general polymers and may show unique properties under specific conditions. The amount of pyruvic acid groups contained at the tail end of the molecular side chain of xanthan gum has a great influence on its performance.

Although the domestic xanthan gum has the advantage in the total value of industrial output, there are still some shortcomings compared with foreign xanthan gum in industrial production technique, such as low gum production rate and unsatisfactory light transmittance of xanthan gum aqueous solution obtained by fermentation. Domestic and foreign studies have shown that the best way to improve the output and quality of xanthan gum products is to transform and screen strains, which has the greatest application value in industrial production. The selection of culture medium components in the xanthan gum fermentation industry is certainly important, but the selection of strains also needs to be carefully considered. The strain is the “chip” of the biological fermentation industry and the decisive factor in product output and quality, which can significantly affect economic benefits, social benefits and ecological benefits. It can be seen that seeking for good strains is extremely important for the xanthan gum industry to further improve the gum production rate and the light transmittance of the xanthan gum aqueous solution obtained by fermentation.

### SUMMARY

The purpose of the present disclosure is to provide Xanthomonas campestris for fermentative production of transparent xanthan gum and use thereof, so as to solve the problems of low gum production rate and unsatisfactory light transmittance of the xanthan gum aqueous solution obtained by fermentation in the existing xanthan gum industry.

To achieve the above purpose, the technical solution adopted by the present disclosure is as follows:

Xanthomonas campestris for fermentative production of transparent xanthan gum, where the Xanthomonas campestris is Xanthomonas campestris F417-6, was deposited in Guangdong Microbial Culture Collection Center on May 12, 2023, with the deposit number of GDMCC NO: 63459 and the deposit address of 5th Floor, Building 59, No. 100 Xianlie Middle Road, Guangzhou.

More preferably, the screening process thereof is as follows:

(1) thawing the bacterial solution of the original Xanthomonas campestris, dipping and streaking the bacterial solution onto the culture medium plate by using an inoculation loop, picking a single colony after the single colony emerges, inoculating into a liquid seed culture medium at an inoculation amount of 1%, culturing at a temperature of 30° C. and 180 r/min in a constant temperature shaker for 24 h, and subculturing twice to restore the original activity of the original Xanthomonas campestris;

(2) inoculating the activated original strain into a 250 ml conical flask containing 100 mL of liquid seed culture medium at an inoculation amount of 1%, culturing at a temperature of 30° C. and 180 r/min in a constant temperature shaker for 24 h to complete the liquid seed culturing;

(3) collecting the bacterial solution of the original Xanthomonas campestris grown to the logarithmic phase and mixing by using a vortex meter to form a uniform bacterial suspension; collecting and placing 1 mL of the prepared bacterial suspension in a 35 mm irradiation dish, sealing with a sealing film, and using a ¹²C⁶⁺ ion beam to perform irradiation mutagenesis, where the ion beam has the extraction energy (energy after extraction) of 80 MeV/u and the LET of 35.5 keV/mm; and performing irradiation with a total of 11 selected mutagenesis doses: 0 Gy, 20 Gy, 40 Gy, 60 Gy, 80 Gy, 100 Gy, 120 Gy, 140 Gy, 160 Gy, 180 Gy, and 200 Gy;

(4) observing the morphologies and sizes of the colonies under different irradiation doses, selecting the relatively large, smooth and transparent colonies respectively and inoculating on a screening culture medium, numbering, and culturing at a constant temperature of 37° C. for 72 h; after the colonies emerge, dripping Lugol's iodine solution to the screening culture medium and observing the transparent circle around each colony; measuring the colony diameter C and the transparent circle diameter H, and calculating the H/C value; and inoculating the strains with larger H/C values into the liquid culture medium, and culturing at a temperature of 30° C. and 180 r/min for 72 h in a constant temperature shaker, where these strains are initial screening strains; and

(5) numbering the initial screening strains, culturing to the logarithmic phase, then inoculating into 75 mL of fermentation culture medium at an inoculation amount of 10% respectively, culturing at 37° C. and 180 r/min for 96 h in a constant temperature shaker, and measuring the gum production rates thereof and the light transmittances of the fermentation products, xanthan gum aqueous solutions, after the fermentation is completed; comparing the gum production rates of the initial screening strains and the light transmittances of the aqueous solutions, selecting optimal strains as the re-screening strains, performing genetic stability test on the re-screening strains, and finally selecting the strain with the best gum production rate, aqueous solution light transmittance, and genetic stability test, as the target strain.

The present disclosure further provides the use of the above-mentioned Xanthomonas campestris as a fermentation strain in the fermentation preparation of xanthan gum.

Specifically, the above use includes the following steps:

(1) strain activation: thawing the preserved bacterial solution containing Xanthomonas campestris with a deposit number of GDMCC NO: 63459, dipping and streaking the preserved bacterial solution onto the culture medium plate by using an inoculation loop, picking a single colony after the single colony emerges and inoculating into a liquid seed culture medium at an inoculation amount of 1%, culturing at a fermentation temperature of 30° C. and 180 r/min for 24 h in a constant temperature shaker, and subculturing twice to restore the original activity of the Xanthomonas campestris;

(2) liquid seed culturing: inoculating the activated Xanthomonas campestris into the liquid seed culture medium at an inoculation amount of 1%, and culturing at a fermentation temperature of 30° C. and 180 r/min for 24 h in a constant temperature shaker to complete the liquid seed culturing;

(3) fermentation culturing: inoculating the seed liquid of Xanthomonas campestris grown to the logarithmic phase into the fermentation culture medium at an inoculation amount of 5-20%, and culturing at a fermentation temperature of 30-40° C. and 180 r/min for 96 h in a constant temperature shaker; and

(4) extracting xanthan gum from the fermentation broth by an ethanol precipitation method.

Further, the culture medium plate in step (1) includes the following components in weight percentage: 0.5% soluble starch, 1% peptone, 0.3% beef extract, 0.5% sodium chloride, and 2% agar. The pH of the culture medium plate is 6.5-7.0, the sterilization temperature is 115° C., the sterilization time is 30 min, and the plate culture time is 72 h.

Further, in step (1) and step (2), the liquid seed culture media each include the following components in weight percentage: 2.0% soluble starch, 0.5% peptone, 0.3% potassium dihydrogen phosphate, and 0.2% sodium chloride. The pH value of the liquid seed culture medium is 7.0.

Further, the inoculation amount in step (3) is 15%.

Further, the fermentation temperature in step (3) is 40° C.

Further, the fermentation culture medium in step (3) includes the following components in weight percentage: 6.0% corn starch, 1.0% glucose, 2.0% soy protein, 0.1% magnesium sulfate heptahydrate, 0.1% dipotassium hydrogen phosphate, and 0.1% potassium dihydrogen phosphate. The pH value of the fermentation culture medium is 7.0.

Compared with the prior art, the present disclosure has the following beneficial effects.

1. For the Xanthomonas campestris (deposit number of GDMCC NO: 63459) provided by the present disclosure, the xanthan gum production rate increases by 15% to 30% compared with the original strain, and the light transmittance of the xanthan gum aqueous solution obtained by fermentation increases by 30% to 50% compared with the original strain.

2. The present disclosure performs genetic stability analysis on the Xanthomonas campestris with the deposit number of GDMCC NO: 63459, and continuous subculturing, measuring the xanthan gum production rate every 2 subcultures. It can be seen that the gum production rate and the gum quality of the xanthan gum of the Xanthomonas campestris provided by the present disclosure do not change significantly after 12 continuous subcultures, proving that it has good stability and may be used as an industrial production strain for large-scale production of highly transparent xanthan gum.

### BRIEF DESCRIPTION OF DRAWINGS

FIG. 1 is a growth curve diagram of the original strain;

FIG. 2 is a line graph showing the relationship between the lethality rate and the irradiation dose of the original strain;

FIG. 3 is a line graph showing the relationship between the positive mutation rate and the irradiation dose of the original strain;

FIG. 4 is a bar analysis chart of the gum production rates for the re-screening strains;

FIG. 5 is a bar analysis chart of light transmittances of fermentation products, xanthan gum aqueous solutions, of the re-screening strains; and

FIG. 6 is a bar analysis chart showing the genetic stability and the gum production rate of Xanthomonas campestris (deposit number of GDMCC NO: 63459) obtained in the present disclosure.

### DETAILED DESCRIPTION OF EMBODIMENTS

The present disclosure will be further described below in conjunction with various embodiments. The embodiments of the present disclosure include but are not limited to the following embodiments.

### Example

The Xanthomonas campestris obtained by the present disclosure (deposit number of GDMCC NO: 63459) was obtained by performing irradiation mutagenesis on the starter strain Xanthomonas campestris through a heavy ion beam irradiation mutagenesis technique, followed by screening and selection. The experimental materials involved in the example were all commercially available, for example, soluble starch, glucose, corn starch, and sodium chloride were purchased from Tianjin Hongyan Chemical Reagent Factory; peptone and soy protein were purchased from Guangdong Huankai Microbiological Technology Co., Ltd.; potassium dihydrogen phosphate and dipotassium hydrogen phosphate were purchased from Tianjin Yongsheng Fine Chemical Co., Ltd.; magnesium sulfate heptahydrate was purchased from Tianjin Baishi Chemical Co., Ltd.; and beef extract was purchased from Beijing Boaoxing Biotechnology Co., Ltd.

The specific mutagenesis technique screening process was as follows:

### 1. Strain Culturing

thawing the bacterial solution of the original Xanthomonas campestris, dipping and streaking a small amount of bacterial solution onto the culture medium plate by using an inoculation loop, picking a single colony after the single colony emerged, inoculating into the liquid seed culture medium, culturing at a temperature of 30° C. and 180 r/min in a constant temperature shaker for 24 hours, inoculating into a 250 ml conical flask containing 100 mL of liquid seed culture medium at an inoculation amount of 1%, culturing at a temperature of 30° C. and 180 r/min in a constant temperature shaker for 24 hours, subculturing twice to restore their original activity, where the original strains were isolated from the leaves of the cruciferous plant cabbage (Brassica oleracea).

The culture medium plate contained 0.5% soluble starch, 1% peptone, 0.3% beef extract, 0.5% sodium chloride, and 2% agar, the pH of the culture medium plate was 6.5-7.0, the sterilization temperature was 115° C., and the sterilization time was 30 min.

### 2. Liquid Seed Culturing

inoculating the activated original strains into a 250 mL conical flask containing 100 mL of liquid seed culture medium at an inoculation amount of 1%, and culturing at 30° C. and 180 r/min in a constant temperature shaker for 24 hours to complete the liquid seed culturing.

The liquid seed culture medium contained 2.0% soluble starch, 0.5% peptone, 0.3% potassium dihydrogen phosphate, and 0.2% sodium chloride, the pH of the seed liquid culture medium was 7.0 and the liquid volume was 100 mL/250 mL.

### 3. Growth Curve Measurement

inoculating the bacterial solution of Xanthomonas campestris which was subjected to subculturing repeatedly for 2-3 times, into 100 ml of liquid seed culture medium at an inoculation amount of 1%, culturing at a temperature of 30° C. and 180 r/min in a constant temperature shaker, measuring the absorbance value of the seed culture medium at 0 h after inoculation at a wavelength of 600 nm, subsequently sampling every 2 hours, measuring the OD₆₀₀ values of the original strains at 0 h, 2 h, 4 h, 8 h, 12 h, 18 h, 24 h, 30 h, 36 h, 48 h, 54h, 60 h, and 72 h, recording the data and plotting the growth curve.

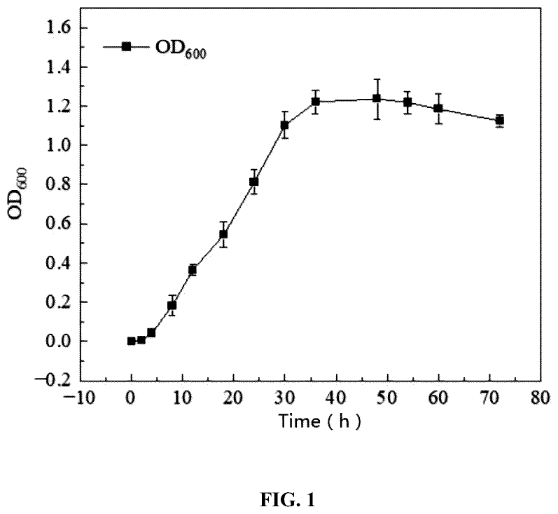

Xanthomonas campestris belongs to Gram-negative pathogenic bacteria, is obligately aerobic, usually rod-shaped with a polar flagellum, and has an appropriate growth temperature range of 25-30° C. The extracellular polysaccharide secreted by Xanthomonas campestris is called xanthan gum, which is a biological gum with excellent performances and may be widely used in many industries with huge market prospects. The fermentation cycle of the original strains is roughly 36-72 h, where bacterial cells are mainly grown and enriched before 36 h, and enter the gum production period after 36 hours. The growth curve of the original strains in liquid culture medium is as shown in FIG. 1. As can be seen from the figure, the growth curve of the original strains conforms to the “S” curve, which conforms to the characteristics of normal growth and reproduction of microbials. The strains were in a growth delay period in 0-8 h, were grown rapidly exponentially in 8-24 h and had OD₆₀₀ reaching 0.813 at 24 h, which was the logarithmic growth period of the strains, then were continuously grown but with decreased growth rate, reached a peak between 36-48 h, which was the stable growth period of the strains, and entered the decline period in the subsequent culture and had the OD₆₀₀ slowly decreased, where the decline of the strains mad the number of bacterial cells in the culture medium reduced.

### 4. Heavy Ion Irradiation Mutagenesis

collecting he bacterial solution of the original strains grown to the logarithmic phase, mixing with a vortex meter to form a uniform bacterial suspension; collecting and placing 1 mL of the prepared bacterial suspension in a 35 mm irradiation dish, sealing with a sealing film, and using the ¹²c⁶⁺ ion beam generated by the Heavy Ion Research Facility in Lanzhou (HIRFL) to perform irradiation mutagenesis, where the ion beam had the extraction energy of 80 MeV/u and the LET of 35.5 keV/mm; and performing irradiation with a total of 11 selected mutagenesis doses: 0 Gy, 20 Gy, 40 Gy, 60 Gy, 80 Gy, 100 Gy, 120 Gy, 140 Gy, 160 Gy, 180 Gy, and 200 Gy;

### 5. Calculation Of Lethality Rate

gradiently diluting the bacterial solutions irradiated with irradiation doses of 0 Gy, 20 Gy, 40 Gy, 60 Gy, 80 Gy, 100 Gy, 120 Gy, 140 Gy, 160 Gy, 180 Gy, and 200 Gy, collecting and spreading 40 μL of the bacterial suspensions on solid culture media, culturing at 37° C. and 180 r/min for 96 h in a constant temperature shaker, where 3 parallel experiments were performed for each group, then recording the number of live bacteria on the plate at different irradiation doses, dividing the number of colonies after irradiation by the number of colonies in the blank control to calculate the lethality rate, and drawing the lethality rate curve with the irradiation dose as the abscissa and the lethality rate as the ordinate:

$$lethality rate ( \% ) = 1 - \frac{number of colonies grown in the irradiation group}{number of colonies grown in the control group} \times 100 \% .$$

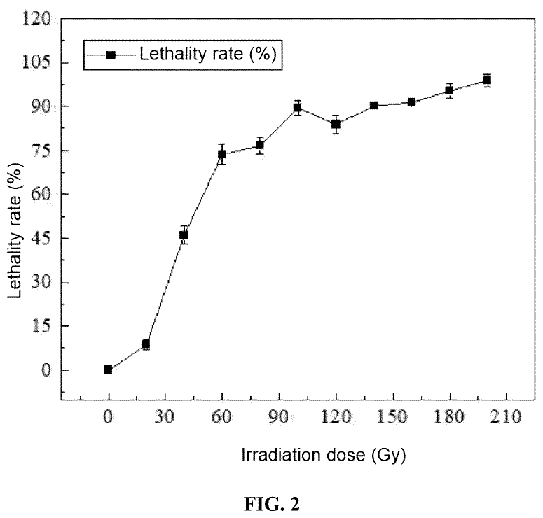

In this experiment, the heavy ion beam was controlled in a stable range, the lethality rate was calculated and the lethality rate curve was drawn by using the relative irradiation dose as the abscissa and the lethality rate as the ordinate. The lethality rates of the original Xanthomonas campestris under different irradiation doses are as shown in Table 1 and FIG. 2. As can be seen from FIG. 2, when the irradiation dose of ¹²C⁶⁺ heavy ions ranges from 0 Gy to 200 Gy, with the increasing of the irradiation dose, the lethality rate curve exhibits an initial increase, followed by a decrease and then gradual increase again, forming a “saddle-shaped” curve. When the irradiation dose is 120 Gy, the lethality rate drops briefly to 83.89%; when the irradiation dose is 140 Gy, the lethality rate rises again and reaches a maximum value of 98.90% at 200 Gy. With the increasing of irradiation dose, the lethality rate of the bacterial cells shows a significant downward trend, forming a concave shape, and this unique “saddle-shaped” curve is considered to be the result of the combined effect of damage effects under the action of energy and momentum, and the protection and stimulation under the actions of mass and charge. With the increasing of the heavy ion irradiation dose, the repair pathway in the strain cells is activated, thereby protecting the DNA from being damaged, so the lethality rate gradually decreases; and with the increasing of the irradiation dose to a certain level, the repair enzymes in the cells are inactivated by heavy ion irradiation, and at this time, the cells are not able to repair themselves correctly, resulting in cell damage or death, so the lethality rate gradually increases again.

TABLE 1
Calculation of Lethality Rate

| Irradiation Dose (Gy) | Lethality Rate (%) |
|---|---|
| 0 | 0 |
| 20 | 8.61 ± 1.82 |
| 40 | 46.14 ± 3.25 |
| 60 | 73.73 ± 3.57 |
| 80 | 76.60 ± 2.85 |
| 100 | 89.62 ± 2.51 |
| 120 | 83.89 ± 3.05 |
| 140 | 90.29 ± 1.15 |
| 160 | 91.39 ± 1.00 |
| 180 | 95.36 ± 2.64 |
| 200 | 98.90 ± 2.08 |

### 6. Calculation of the Positive Mutation Rate

appropriately diluting the bacterial suspension of the original Xanthomonas campestris mutagenized under different irradiation doses, collecting and spreading 0.1 mL on the solid culture medium, culturing at 37° C. for 48 h, observing the morphologies and sizes of the colonies under different irradiation doses, picking and inoculating relatively large colonies on the screening culture medium, culturing at 37° C. for 72 h; after the colonies emerged, dripping Lugol's iodine solution onto the screening culture medium, observing the transparent circles around the colonies; measuring the colony diameter (C) and the transparent circle diameter (H), and calculating the positive mutation rate according to the formula below, drawing the positive mutation rate curve with the irradiation time as the abscissa and the positive mutation rate as the ordinate, selecting the strains of the colonies with relatively large transparent circle around and inoculating into the liquid culture medium, culturing at 37° C. and 180 r/min in a constant temperature shaker for 24 h, and then storing for subsequent fermentation screening,

$$positive mutation rate ( \% ) = \frac{n umber of colonies where \frac{H}{C} value of mutant strain is increased by 20 \% or more compared to the original strain}{total number of colonies in the experiment} \times 100 \% .$$

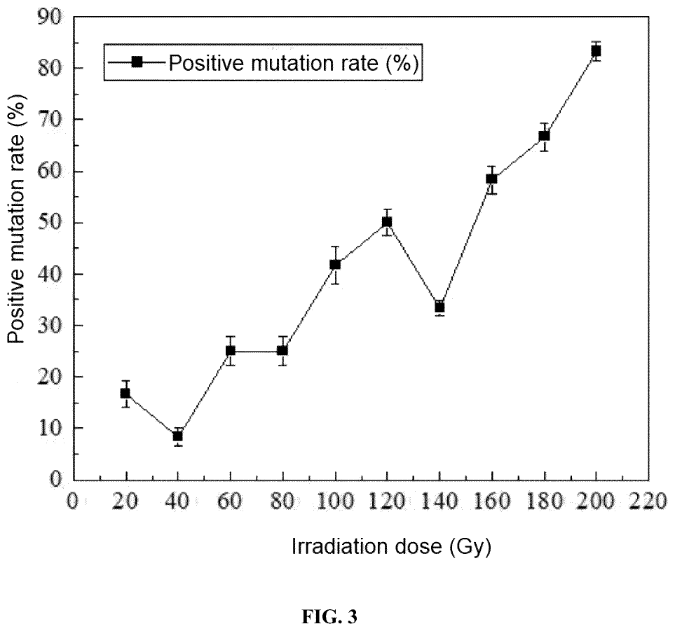

The positive mutation rate of mutagenesis of original Xanthomonas campestris was calculated according to the ratio of the transparent circle diameter to the colony diameter; and the positive mutation rate was calculated and a positive mutation rate curve was drawn by using the relative irradiation dose as the abscissa and the positive mutation rate as the ordinate. The positive mutation rates of Xanthomonas campestris under different irradiation doses are as shown in Table 2 and FIG. 3. As can be seen in FIG. 3, when the irradiation dose of ¹²c⁶⁺ heavy ions is within 0 Gy-200 Gy, with the irradiation dose increasing, the positive mutation rate curve exhibits an initial decrease, followed by an increase, and then decrease again and then increase, where when the irradiation dose is 40 Gy, the positive mutation rate reaches the lowest, which is 8.33%; when the irradiation dose is 40-200 Gy, the positive mutation rate shows an “increase-decrease-increase” trend and has a decrease point appearing at 140 Gy, which is 33.33%; and when the irradiation dose is 200 Gy, the positive mutation rate is the highest, which is 83.33%, and the H/C value is the largest at this time. It means that the greater the irradiation dose, the higher the positive mutation rate, the more severe the damage to the DNA in the bacterial cells, and the higher the possible mutation rate of the strain. Under high irradiation doses, the internal structures of the cell, such as the structure of substances such as DNA and protein, are destroyed, and the genetic information varies, the bacteria are more likely to mutate, and positive mutations are also very likely to occur, which is conducive to screening out high-producing strains from the mutagenized strains.

TABLE 2
Calculation of Positive Mutation Rate

| Irradiation dose (Gy) | Positive mutation rate |
|---|---|
| 0 | 0 |
| 20 | 16.67 ± 2.54 |
| 40 | 8.33 ± 1.65 |
| 60 | 25.33 ± 2.78 |
| 80 | 25.33 ± 2.75 |
| 100 | 41.67 ± 3.67 |
| 120 | 50.67 ± 2.54 |
| 140 | 33.33 ± 1.54 |
| 160 | 58.33 ± 2.65 |
| 180 | 66.67 ± 2.76 |
| 200 | 83.33 ± 1.87 |

### 7. Initial Screening of Mutagenized Strains

observing the morphologies and sizes of the colonies under different irradiation doses, picking relatively large, smooth and transparent colonies respectively, inoculating on the screening culture medium, numbering, and culturing at a constant temperature of 37° C. for 72 h; after the colonies emerged, dripping Lugol's iodine solution to the screening culture medium, observing the transparent circle around each colony, measuring the colony diameter (C) and the transparent circle diameter (H), calculating the H/C value, inoculating strains with larger H/C values into the liquid culture medium and culturing at a temperature of 30° C. and 180 r/min in a constant temperature shaker for 72 h, where these strains are the initial screening strains.

The results of H/C values, after inoculation, of colonies picked from the original Xanthomonas campestris at different irradiation doses are as shown in Table 3.

TABLE 3
Results of H/C value of original Xanthomonas campestris

| Strain number | H/C value |
|---|---|
| F-0-1 | 4.70 |
| F-0-2 | 4.37 |
| F-0-3 | 4.86 |
| F-0-4 | 3.93 |
| F-0-5 | 4.27 |
| F-0-6 | 3.84 |
| F-0-7 | 4.80 |
| F-0-8 | 4.24 |
| F-0-9 | 4.62 |
| F-0-10 | 4.02 |
| F-0-11 | 4.63 |
| F-0-12 | 4.38 |
| F-20-1 | 7.83 |
| F-20-2 | 4.15 |
| F-20-3 | 4.51 |
| F-20-4 | 3.90 |
| F-20-5 | 4.62 |
| F-20-6 | 4.41 |
| F-20-7 | 7.25 |
| F-20-8 | 4.69 |
| F-20-9 | 4.91 |
| F-20-10 | 4.72 |
| F-20-11 | 4.80 |
| F-20-12 | 7.53 |
| F-40-1 | 4.82 |
| F-40-2 | 4.23 |
| F-40-3 | 4.36 |
| F-40-4 | 7.33 |
| F-40-5 | 4.74 |
| F-40-6 | 3.50 |
| F-40-7 | 4.12 |
| F-40-8 | 4.11 |
| F-40-9 | 4.41 |
| F-40-10 | 4.29 |
| F-40-11 | 3.98 |
| F-40-12 | 4.24 |
| F-60-1 | 2.37 |
| F-60-2 | 3.37 |
| F-60-3 | 2.34 |
| F-60-4 | 2.80 |
| F-60-5 | 7.05 |
| F-60-6 | 2.11 |
| F-60-7 | 3.41 |
| F-60-8 | 3.48 |
| F-60-9 | 3.86 |
| F-60-10 | 4.18 |
| F-60-11 | 7.77 |
| F-60-12 | 7.90 |
| F-80-1 | 4.10 |
| F-80-2 | 7.19 |
| F-80-3 | 3.50 |
| F-80-4 | 3.69 |
| F-80-5 | 3.75 |
| F-80-6 | 3.59 |
| F-80-7 | 4.25 |
| F-80-8 | 3.98 |
| F-80-9 | 4.86 |
| F-80-10 | 2.85 |
| F-80-11 | 3.06 |
| F-80-12 | 3.31 |
| F-100-1 | 4.24 |
| F-100-2 | 4.39 |
| F-100-3 | 4.70 |
| F-100-4 | 4.88 |
| F-100-5 | 5.54 |
| F-100-6 | 5.58 |
| F-100-7 | 6.50 |
| F-100-8 | 5.18 |
| F-100-9 | 5.62 |
| F-100-10 | 4.20 |
| F-100-11 | 7.06 |
| F-100-12 | 5.39 |
| F-120-1 | 7.14 |
| F-120-2 | 6.88 |
| F-120-3 | 5.23 |
| F-120-4 | 7.82 |
| F-120-5 | 5.13 |
| F-120-6 | 5.17 |
| F-120-7 | 4.90 |
| F-120-8 | 6.32 |
| F-120-9 | 5.33 |
| F-120-10 | 5.46 |
| F-120-11 | 5.50 |
| F-120-12 | 6.31 |
| F-140-1 | 5.56 |
| F-140-2 | 5.92 |
| F-140-3 | 7.13 |
| F-140-4 | 5.61 |
| F-140-5 | 4.87 |
| F-140-6 | 5.68 |
| F-140-7 | 5.44 |
| F-140-8 | 4.83 |
| F-140-9 | 5.68 |
| F-140-10 | 4.71 |
| F-140-11 | 5.22 |
| F-140-12 | 7.69 |
| F-160-1 | 6.03 |
| F-160-2 | 5.45 |
| F-160-3 | 5.61 |
| F-160-4 | 5.28 |
| F-160-5 | 5.31 |
| F-160-6 | 6.44 |
| F-160-7 | 6.10 |
| F-160-8 | 6.38 |
| F-160-9 | 7.42 |
| F-160-10 | 6.73 |
| F-160-11 | 7.11 |
| F-160-12 | 5.72 |
| F-180-1 | 7.63 |
| F-180-2 | 5.50 |
| F-180-3 | 6.00 |
| F-180-4 | 6.00 |
| F-180-5 | 7.13 |
| F-180-6 | 5.59 |
| F-180-7 | 5.93 |
| F-180-8 | 5.66 |
| F-180-9 | 6.05 |
| F-180-10 | 6.39 |
| F-180-11 | 7.20 |
| F-180-12 | 6.10 |
| F-200-1 | 5.50 |
| F-200-2 | 6.48 |
| F-200-3 | 7.16 |
| F-200-4 | 7.37 |
| F-200-5 | 7.32 |
| F-200-6 | 5.80 |
| F-200-7 | 6.29 |
| F-200-8 | 8.09 |
| F-200-9 | 5.87 |
| F-200-10 | 6.13 |
| F-200-11 | 6.28 |
| F-200-12 | 8.65 |

The H/C values of strains F-0-1 to F-0-12 are the same as those of the original Xanthomonas campestris. As can be seen from Table 3, after heavy ion irradiation mutagenesis of the original Xanthomonas campestris, the transparent circles of most colonies became larger and the H/C values increased, indicating that their ability to use starch was improved, the H/C values of some colonies decreased, indicating a negative mutation, and by comparing the magnitudes of the H/C values, a total of 23 strains were obtained in the initial screening and named according to the irradiation doses of the strains, respectively namely F-20-1, F-20-7, F-20-12, F-40-4, F-60-5, F-60-11, F-60-12, F-80-2, F-100-11, F-120-1, F-120-4, F-140-3, F-140-12, F-160-9, F-160-11, F-180-1, F-180-5, F-180-11, F-200-3, F-200-4, F-200-5, F-200-8, and F-200-12. The 23 mutant strains were cultured to the logarithmic phase, inoculated into the fermentation culture medium at an inoculation amount of 5%, and cultured at a constant temperature of 37° C. and 180 r/min in a shaker for 96 h, the xanthan gum production rate was measured, the fermentation product xanthan gum was dissolved in deionized water to prepare a solution with a concentration of 2.0 g/L, and the light transmittance of the aqueous solution was measured. The results of the xanthan gum production rate and the light transmittance of the aqueous solution are as shown in Table 4.

TABLE 4
Initial Screening Results

| | Gum | |
|---|---|---|
| Strain | production | Light |
| number | rate /% | transmittance/% |
| F-0 | 3.30 ± 0.04 | 65.87 ± 0.99 |
| F-20-1 | 2.05 ± 0.07 | 72.76 ± 1.54 |
| F-20-7 | 2.94 ± 0.07 | 78.53 ± 1.06 |
| F-20-12 | 3.72 ± 0.11 | 95.07 ± 1.25 |
| F-40-4 | 3.32 ± 0.13 | 97.21 ± 1.57 |
| F-60-5 | 2.76 ± 0.15 | 85.87 ± 1.53 |
| F-60-11 | 1.94 ± 0.11 | 90.54 ± 1.83 |
| F-60-12 | 2.45 ± 0.11 | 97.34 ± 1.03 |
| F-80-2 | 2.54 ± 0.03 | 78.45 ± 1.00 |
| F-100-11 | 2.73 ± 0.24 | 97.65 ± 1.22 |
| F-120-1 | 2.56 ± 0.09 | 72.42 ± 0.98 |
| F-120-4 | 2.88 ± 0.03 | 66.43 ± 1.61 |
| F-140-3 | 3.86 ± 0.04 | 94.34 ± 2.03 |
| F-140-12 | 2.94 ± 0.08 | 76.56 ± 1.75 |
| F-160-9 | 4.26 ± 0.16 | 98.41 ± 1.11 |
| F-160-11 | 3.76 ± 0.07 | 96.32 ± 1.43 |
| F-180-1 | 3.28 ± 0.18 | 97.81 ± 1.57 |
| F-180-5 | 2.92 ± 0.08 | 87.40 ± 0.93 |
| F-180-11 | 2.47 ± 0.06 | 94.56 ± 1.53 |
| F-200-3 | 2.04 ± 0.17 | 65.22 ± 1.87 |
| F-200-4 | 2.45 ± 0.12 | 68.75 ± 1.00 |
| F-200-5 | 1.69 ± 0.07 | 67.09 ± 1.57 |
| F-200-8 | 2.41 ± 0.12 | 75.88 ± 1.12 |
| F-200-12 | 2.99 ± 0.06 | 72.53 ± 1.58 |

### 8. Re-screening of Mutagenized Strains

numbering the initial screening strains, culturing to the logarithmic phase, inoculating into 75 mL of fermentation culture medium at an inoculation amount of 10%, culturing at a temperature of 37° C. and 180 r/min for 96 h in a constant temperature shaker, measuring the gum production rate thereof and the light transmittance of the fermentation product xanthan gum aqueous solution after the fermentation; comparing the gum production rates and light transmittances of the aqueous solutions of the initial screening strains, selecting the optimal strains as the re-screening strains, and testing the genetic stability of the re-screening strains.

In the above, the xanthan gum extraction and the calculation process of the gum production rate and aqueous solution light transmittance of the strain were as follows:

extraction of xanthan gum: when the OD₆₀₀ value of the liquid seed was 0.8, inoculating the seed liquid of the target strain grown to the logarithmic phase into a 250 mL conical flask containing 100 mL of fermentation culture medium at an inoculation amount of 15%, culturing at a constant temperature of 40° C. and 180 r/min in a shaker for 96 h, using the ethanol precipitation method: adding 3 times of the volumes of deionized water to the fermentation broth to dilute the fermentation broth, and then adding 3 times of the volumes of anhydrous ethanol to precipitate xanthan gum, centrifuging at 8,000 r/min for 15 min, discarding the supernatant and retaining the precipitates, washing the precipitates twice with anhydrous ethanol, drying in a 60° C. oven, grinding to obtain the fermentation product xanthan gum, and weighting,

where the liquid fermentation culture medium contained 6.0% corn starch, 1.0% glucose, 2.0% soy protein, 0.1% magnesium sulfate heptahydrate, 0.1% dipotassium hydrogen phosphate, and 0.1% potassium dihydrogen phosphate, the pH of the liquid fermentation culture medium was 7.0, and the liquid volume was 75 mL/250 ml;

calculation of gum production rate: dividing the mass of extracted xanthan gum by the mass of fermentation broth to give the xanthan gum production rate;

$$xanthan gum production rate ( \% ) = \frac{the mass of xanthan gum sample ( g )}{the mass of xanthan gum fermentation broth ( g )} \times 100 \% .$$

calculation of light transmittance: dissolving the fermentation product xanthan gum in deionized water to prepare a solution with a concentration of 2.0 g/L, and centrifuging at 8,000 rpm for 10 min to remove bubbles; using a ultraviolet-visible spectrophotometer to measure the absorbance of the aqueous solution at 600 nm, with the deionized water used as a blank control, where 3 parallel experiments were performed for each group, recording the data, and calculating the light transmittance of the fermentation product xanthan gum aqueous solution according to the following formula:

$$A = {log}_{10} \frac{1}{T}$$

A—absorbance of sample;

T—transmissivity of the sample.

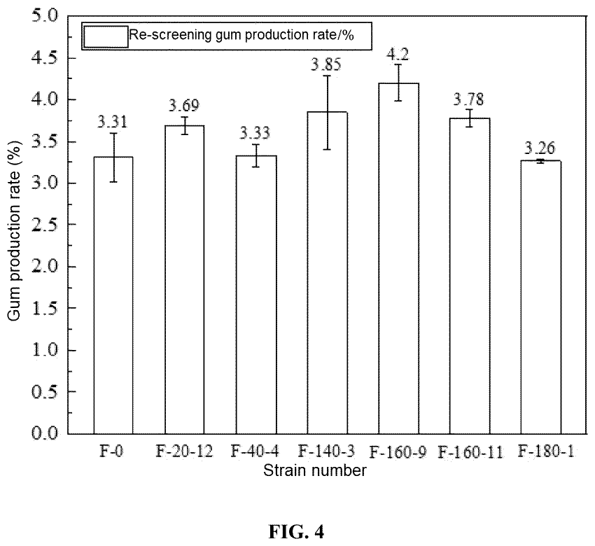

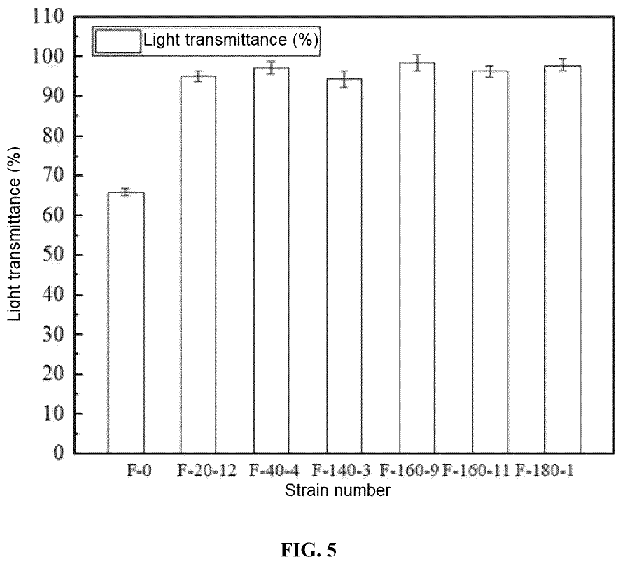

After initial screening and comparison of the gum production rates and light transmittances of the initial screening strains, six re-screening mutant strains F-20-12, F-40-4, F-140-3, F-160-9, F-160-11, and F-180-1 were determined. The gum production rates and light transmittances were measured by fermentation. The results are as shown in Table 5, FIG. 4 and FIG. 5. As seen from FIG. 4 and FIG. 5, the original Xanthomonas campestris has a gum production rate of 3.31% and an aqueous solution light transmittance of 65.87%. For F-160-9, the gum production rate is 4.20%, which is 26.89% higher than that of the original Xanthomonas campestris; the aqueous solution light transmittance is 98.41%, which is 49.40% higher than that of the original Xanthomonas campestris. The xanthan gum production rate and aqueous solution light transmittance of the Xanthomonas campestris after heavy ion irradiation mutagenesis are significantly improved, and compared with the initial screening results, the H/C value has a certain positive correlation with the xanthan gum production rate, indicating that the initial screening results are reliable. By comparing the gum production rates and light transmittances of the 6 re-screening strains, F-160-9 is finally determined to be the mutant strain obtained by the final screening, indicating that a mutagenized strain for xanthan gum production with higher gum production rate and stronger light transmittance of xanthan gum aqueous solution may be obtained by performing mutagenesis on the original Xanthomonas campestris using heavy ion beams, and the mutagenized strain cultured after heavy ion irradiation mutagenesis has positive mutation. The mutant strain F-160-9 is preserved, with the biological material name and identification characteristic of Xanthomonas campestris F417-6; in the preservation unit Guangdong Microbial Culture Collection Center, the address of which is 5th Floor, Building 59, No. 100 Xianlie Middle Road, Guangzhou, the deposit number is GDMCC NO: 63459, and the deposit date is May 12, 2023.

TABLE 5
Re-Screening Results

| Strain number | Gum production rate/% | Light Transmittance/% |
|---|---|---|
| F-0 | 3.31 ± 0.34 | 65.87 ± 0.99 |
| F-20-12 | 3.69 ± 0.11 | 95.07 ± 1.25 |
| F-40-4 | 3.33 ± 0.13 | 97.21 ± 1.57 |
| F-140-3 | 3.85 ± 0.44 | 94.34 ± 2.03 |
| F-160-9 | 4.20 ± 0.21 | 98.41 ± 1.11 |
| F-160-11 | 3.78 ± 0.10 | 96.32 ± 1.43 |
| F-180-1 | 3.26 ± 0.02 | 97.81 ± 1.57 |

### 9. Genetic Stability Determination

performing genetic performance stability tests on the screened mutagenized strains, continuously subculturing for 12 times under the same culture conditions, observing the xanthan gum production rate and light transmittance of the aqueous solution after subculturing, and determining whether they had genetic stability.

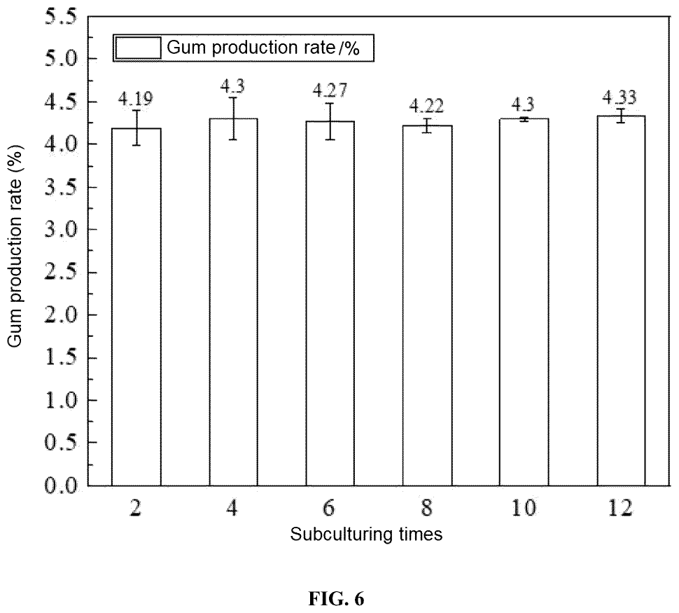

The mutant strain Xanthomonas campestris (deposit number of GDMCC NO: 63459) was subjected to subculturing for 12 times under the same conditions, and inoculated into a liquid fermentation culture medium every two subcultures to measure the gum production rate (FIG. 6) and the light transmittance of xanthan gum aqueous solution (Table 6), where the differences in the gum production rate and light transmittance were not large under the analysis of standard deviation, indicating that the genetic performance of this high-producing strain Xanthomonas campestris was stable. By using the physical mutagenesis means of heavy ion mutagenesis to perform artificial breeding, the experiment obtained a mutant strain with stable performance and not prone to recovery, and may be considered as an industrial production strain for the scale-up production of highly transparent xanthan gum.

TABLE 6
Light Transmittance Genetic Stability

| Subculture Generation | Light Transmittance/% |
|---|---|
| 2 | 98.54 ± 1.77 |
| 4 | 98.21 ± 1.34 |
| 6 | 98.33 ± 1.61 |
| 8 | 98.21 ± 1.76 |
| 10 | 98.32 ± 1.32 |
| 12 | 98.80 ± 1.54 |

The above example is only one of the preferred embodiments of the present disclosure and should not be used to limit the protection scope of the present disclosure. Any changes or modifications, that are made to the main design concept and spirit of the present disclosure, have no substantive significance, and solve the technical problems still consistent with the present disclosure, should be included in the protection scope of the present disclosure.

1. Xanthomonas campestris for fermentative production of transparent xanthan gum, wherein the Xanthomonas campestris is Xanthomonas campestris F417-6, and deposited in Guangdong Microbial Culture Collection Center on May 12, 2023, with a deposit number of GDMCC NO: 63459, and a deposit address is: 5th Floor, Building 59, No. 100 Xianlie Middle Road, Guangzhou.

2. A method for using the Xanthomonas campestris according to claim 1, comprising preparing xanthan gum by performing fermentation using the Xanthomonas campestris as a fermentation strain.

3. The method according to claim 2, comprising following steps:
(1) strain activation: thawing a preserved bacterial solution containing the Xanthomonas campestris, dipping and streaking the preserved bacterial solution onto a culture medium plate by using an inoculation loop, picking a single colony after the single colony emerges, inoculating into a liquid seed culture medium at an inoculation amount of 1%, culturing at a fermentation temperature of 30° C. and 180 r/min for 24 h in a constant temperature shaker, and subculturing twice to restore an original activity of the Xanthomonas campestris;
(2) liquid seed culturing: inoculating activated Xanthomonas campestris into a liquid seed culture medium at an inoculation amount of 1%, and culturing at a fermentation temperature of 30° C. and 180 r/min for 24 h in the constant temperature shaker to complete the liquid seed culturing;
(3) fermentation culturing: inoculating a seed liquid of the Xanthomonas campestris grown to a logarithmic phase into a fermentation culture medium at an inoculation amount of 5-20%, and performing constant-temperature culturing at a fermentation temperature of 30-40° C. and 180 r/min in a shaker for 96 h; and
(4) extracting the xanthan gum from a fermentation broth by using an ethanol precipitation method.

4. The method according to claim 3, wherein the culture medium plate in the step (1) comprises following components in weight percentage: 0.5% soluble starch, 1% peptone, 0.3% beef extract, 0.5% sodium chloride, and 2% agar, a pH of the culture medium plate is 6.5-7.0, a sterilization temperature is 115° C., a sterilization time is 30 min, and a plate culture time is 72 h.

5. The method according to claim 3, wherein in the step (1) and the step (2), the liquid seed culture medium comprises following components in weight percentage: 2.0% soluble starch, 0.5% peptone, 0.3% potassium dihydrogen phosphate, and 0.2% sodium chloride, and a pH value of the liquid seed culture medium is 7.0.

6. The method according to claim 3, wherein the inoculation amount in the step (3) is 15%.

7. The method according to claim 3, wherein the fermentation temperature in the step (3) is 40° C.

8. The method according to claim 3, wherein the fermentation culture medium in the step (3) comprises following components in weight percentage: 6.0% corn starch, 1.0% glucose, 2.0% soy protein, 0.1% magnesium sulfate heptahydrate, 0.1% dipotassium hydrogen phosphate, and 0.1% potassium dihydrogen phosphate, and a pH value of the fermentation culture medium is 7.0.
